(19) United States
(12) Patent Application Publication
(10) Pub. No.: US 2025/0134841 A1
(43) Pub. Date: May 1, 2025

(54) PHARMACEUTICAL COMPOSITION OF BEMPEDOIC ACID

(71) Applicant: Synthon B.V., Nijmegen (NL)

(72) Inventors: Marta VIVANCOS MARTINEZ, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 18/690,975

(22) PCT Filed: Sep. 12, 2022
(86) PCT No.: PCT/EP2022/075272

§ 371 Date: Mar. 11, 2024

(30) Foreign Application Priority Data
Sep. 13, 2021 (EP) 21196350.9

(51) Int. Cl. A61K 31/20; A61K 9/20

(52) U.S. Cl. CPC A61K 31/20; A61K 9/2009; A61K 9/2013; A61K 9/2018; A61K 9/2027; A61K 9/2054; A61K 9/2059

(57) ABSTRACT

The presented invention relates to pharmaceutical composition comprising Bempedoic acid and processes for preparation thereof.

The invention relates to a pharmaceutical composition comprising Bempedoic acid, compound of formula (1), and to a process for preparation thereof.

### BACKGROUND OF THE PRESENT INVENTION

This invention relates to a pharmaceutical composition comprising Bempedoic acid, compound of formula (1) and to a process for preparation thereof;

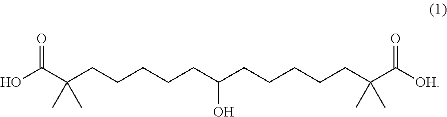 [SMILES: CC(C)(CCCCCC(O)CCCCCC(C)(C)C(=O)O)C(=O)O]

Bempedoic acid, 8-Hydroxy-2,2,14,14-tetramethylpentadecanedioic acid, is a dual-acting AMP-activated protein kinase (AMPK) activator and ATP citrate lyase (ACL) inhibitor. Bempedoic acid has been approved for the oral treatment of hypercholesterolemia. In Europe, it is marketed as a tablet under the brand name NILEMDO® (Daiichi Sankyo Europe GmbH); in US under the brand name NEXLETOL® (Esperion Therapeutics, Inc.).

Bempedoic acid was first described in patent application WO2004067489. Pharmaceutical compositions comprising Bempedoic acid have been described in WO18218147 and WO2019161307. Due to the high load of BPA in the pharmaceutical composition, BPA properties (low melting point, poor flow and stickiness) have a high impact on the manufacturing process and final drug product features. WO2019161307 improves the bioavailability and pharmacokinetic characteristics of Bempedoic acid preparing sustained-release compositions comprising Bempedoic acid. WO18218147 solves the poor flow characteristics and stickiness of Bempedoic acid avoiding standard granulation process and performing a long pre-blending (at least 45 minutes) with a lubricant (e.g. colloidal silicon dioxide, sodium stearyl fumarate, magnesium stearate). There is still a need of alternative and improved formulations that mitigate the impact of the poor drug substance properties, in particular stickiness on the manufacturing process and drug product quality. It is therefore desirable to develop a simple and stable pharmaceutical composition of Bempedoic acid which overcome the problems of the prior art and which is advantageously manufactured and is bioequivalent to the commercial Bempedoic acid tablet NILEMDOR/NEXLETOL®.

### BRIEF DESCRIPTION OF THE INVENTION

The presented invention relates to a pharmaceutical composition comprising Bempedoic acid, compound of formula (1) and magnesium aluminometasilicate and to a process for preparation thereof;

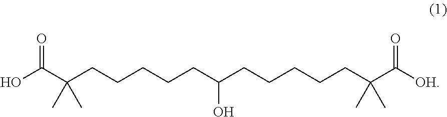 [SMILES: CC(C)(CCCCCC(O)CCCCCC(C)(C)C(=O)O)C(=O)O]

The pharmaceutical composition of the present invention is simple, stable, it is advantageously manufactured and mimics the dissolution profile of the commercial Bempedoic acid tablet NILEMDOR/NEXLETOL®.

### BRIEF DESCRIPTION OF THE DRAWINGS

FIG. 1 depicts the dissolution profiles of compositions according to Examples 1, 2 and 3.

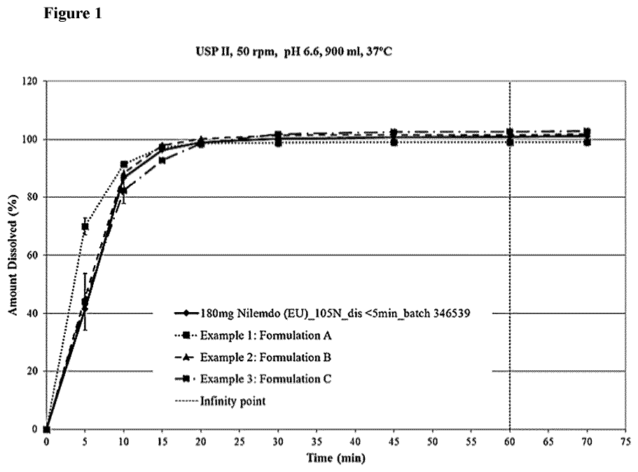

FIG. 2 depicts the process according to Example 1.

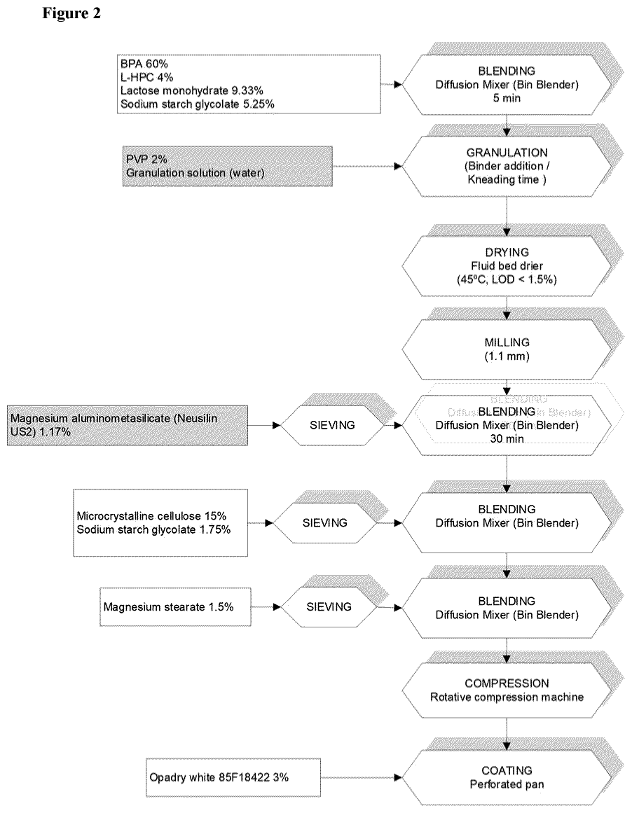

FIG. 3 depicts the process according to Example 2.

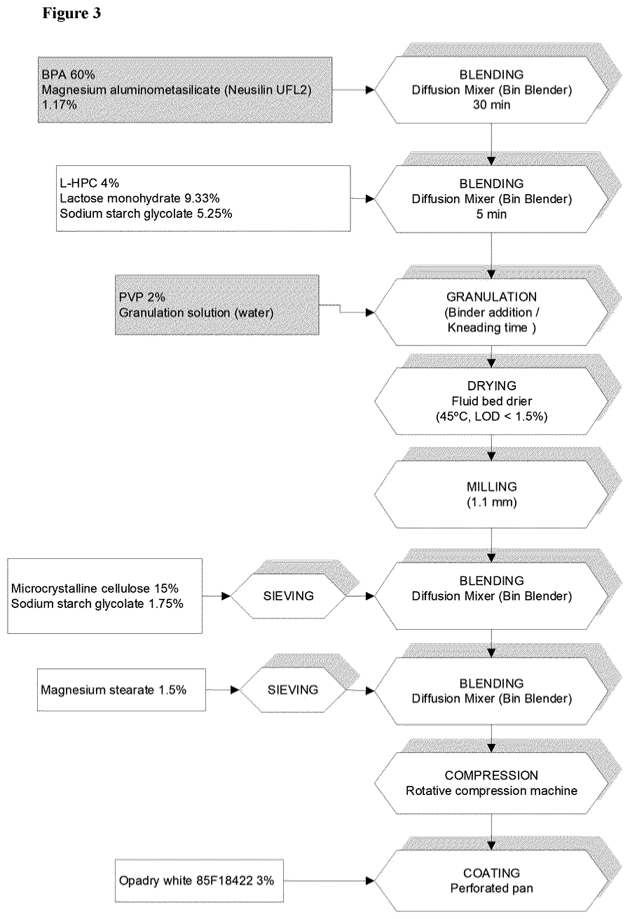

FIG. 4 depicts the process according to Example 3.

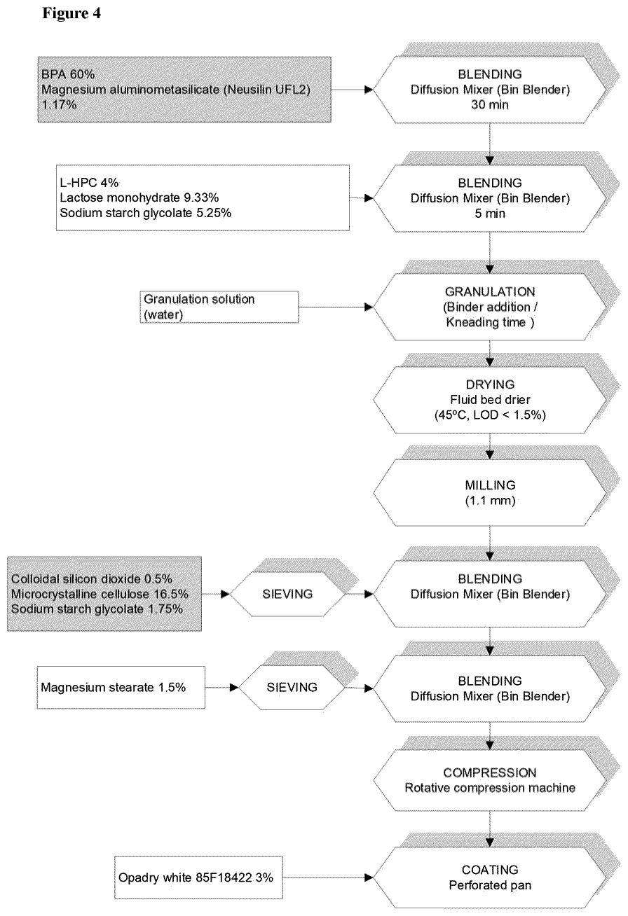

### DETAILED DESCRIPTION OF THE INVENTION

The presented invention relates to a pharmaceutical composition comprising Bempedoic acid and magnesium aluminometasilicate.

Magnesium aluminometasilicate within this invention encompass any synonyms such as silodrate hydrate, simaldrate and aluminum magnesium oxide silicate.

Bempedoic acid presents poor flow and sticky bulk properties and it is therefore difficult to formulate, especially in steps as granulation, blending and compression.

These problems have a negative impact on the drug manufacturing process such that common operations as granulation or tablet compression cannot be performed.

The inventors have found that surprisingly, the presence of magnesium aluminometasilicate in formulations comprising Bempedoic acid reduces the sticky behavior of Bempedoic acid not only when pre-blended with Bempedoic acid, but also when added extragranularly and blended with Bempedoic acid granules. Moreover, a long pre-blend of magnesium aluminometasilicate with Bempedoic acid is not needed, reducing process time and cost in the commercial scale. Magnesium aluminometasilicate not only prevents stickiness but also prevents Bempedoic acid melting during the compression process. Hence, the use of this excipient in a tablet composition prevents the formation of “flakes” which is melt material formed during the compression that jeopardize this step; reducing or eliminating this problem. Typically, the skilled person will adjust the compression speed to maintain the tablet quality. Magnesium aluminometasilicate improves tabletting speed. Pharmaceutical compositions comprising Bempedoic acid and magnesium aluminometasilicate of the current invention are simple to prepare, are stable, and mimic the dissolution profile of the commercial Bempedoic acid tablet NILEMDOR/NEXLETOL®.

In a first embodiment, the present invention relates to a pharmaceutical composition comprising Bempedoic acid and magnesium aluminometasilicate wherein the weight ratio of magnesium aluminometasilicate to Bempedoic acid ranges from 1:2 to 1:120. A more preferred range is from 1:20 to 1:80, even more preferred range is from 1:30 to 1:70, a most preferred range is from 1:40 to 1:60.

Particularly, the pharmaceutical composition of the invention comprises a therapeutically effective dose of Bempedoic acid having a particle size distribution D90 from 3 to 100 μm, preferably from 3 to 50 μm, more preferred from 3 to 40 μm. The D90 value of the particle size distribution is defined as the particle diameter at which 90% by volume of the particles have a smaller diameter than the diameter which corresponds to the D90 value measured by laser diffractometry. Specifically, a Malvern Instruments Mastersizer was used to determine the particle size distribution.

Further, the present invention provides for a pharmaceutical composition wherein Bempedoic acid is present in an amount of from 20% to 80%, preferably from 30% to 70%, more preferably from 40% to 70% by weight based on the total composition weight.

In a preferred embodiment of the present invention Bempedoic acid is present in an amount of 60% by weight based on the total composition weight.

Moreover, the present invention provides for a pharmaceutical composition wherein magnesium aluminometasilicate is present in an amount of from 0.5% to 25%, preferably from 0.5% to 15%, more preferably from 0.5% to 10%, even more preferably from 0.5% to 5% by weight based on the total composition weight.

In a first embodiment, the pharmaceutical composition as described above is manufactured by granulation and comprises the magnesium aluminometasilicate intragranularly and/or extragranularly.

Besides magnesium aluminometasilicate one or more pharmaceutically acceptable excipients can be used additionally in accordance with the present invention. The excipients can be used only intragranularly, only extragranularly or both.

The one or more pharmaceutically acceptable excipients to be used additionally to magnesium aluminometasilicate in accordance with the present invention can be chosen from, for example, fillers, binders, disintegrants, lubricants and glidants.

Fillers are used to increase the bulk volume of a tablet or capsule. By combining a filler with the active pharmaceutical ingredient, the final product is given adequate weight and size to assist in production and handling. The pharmaceutical composition of the present invention preferably comprises at least one filler.

Fillers are preferably used in an amount of from 5% to 70%, more preferably of from 10% to 50%, even more preferably of from 10% to 40%, most preferably of from 10% to 30% by weight based on the total weight of the composition. Suitable examples of fillers to be used in accordance with the present invention include mannitol, sorbitol, microcrystalline cellulose, lactose, lactose monohydrate, phosphates, cellulose, hydroxypropyl cellulose, starch, pregelatinized starch, modified starch, sucrose, dextrose, dextrates, maltodextrin, xylitol, cyclodextrines, calcium phosphate, calcium sulfate and talc.

In a preferred embodiment of the present invention, the fillers to be used are microcrystalline cellulose, lactose monohydrate or mixtures thereof.

The pharmaceutical composition of the present invention may also comprise one or more binders. Binders ensure that tablets and granules can be formed having the desired or required mechanical strength. Binders which are suitable for use in accordance with the present invention include povidone, low substituted hydroxypropyl cellulose, hydroxypropyl methylcellulose, hydroxymethylpropylcellulose, sodium carboxyl methylcellulose, pregelatinized starch, starch, PEG and gelatin. Binders are preferably used in an amount of from 0.5% to 8%, and more preferred 1% to 6% by weight based on the total weight of the composition.

In a preferred embodiment of the present invention, the binder to be used comprises hydroxypropylcellulose.

The pharmaceutical composition of the present invention may also comprise one or more disintegrants. Disintegrants are added to a tablet or capsule composition to promote the breakup of the tablet/capsule into smaller fragments in an aqueous environment, thereby increasing the available surface area and promoting a more rapid release of the active pharmaceutical ingredient. Suitable examples of disintegrants to be used in accordance with the present invention include crospovidone, sodium starch glycolate, croscarmellose sodium, natural starch, pregelatinized starch, sodium starch, methylcrystalline cellulose, methylcellulose, croscarmellose, crosslinked sodium carboxymethylcellulose, cross-linked croscarmellose, cross-linked polyvinilpyrrolidone, sodium alginate and gum. Disintegrants are preferably used in an amount of from 1% to 15% by weight, more preferably of from 2% to 10%, even more preferably of from 5% to 10% by weight based on the total weight of the composition.

In a preferred embodiment of the present invention, the disintegrant to be used is sodium starch glycolate.

The pharmaceutical composition of the invention may also comprise one or more lubricants. Lubricants are generally used in order to reduce sliding friction. In particular, to decrease the friction at the interface between the blend and the compression machine.

Suitable lubricants to be used in accordance with the present invention include magnesium stearate, calcium stearate, stearic acid, glyceryl behenate, hydrogenated vegetable oil, talc and sodium stearyl fumarate. Lubricants are preferably used in an amount of from 0.5% to 10% by weight, more preferably of from 1% to 5%, even more preferably of from 1% to 2% by weight based on the total weight of the composition. In a preferred embodiment of the present invention, the lubricant to be used is magnesium stearate.

The pharmaceutical composition of the present invention may also optionally comprise one or more glidants. Glidants enhance product flow by reducing interparticulate friction. A suitable example in accordance with the present invention is colloidal silicon dioxide. Glidants are preferably used in an amount of from 0.2% to 10% by weight, more preferably of from 0.2% to 5%, even more preferably of from 0.2% to 2% by weight based on the total weight of the composition.

In a first preferred embodiment, the pharmaceutical composition of the present invention contains the following ingredients, based on the total weight of the composition:

a. Bempedoic acid in an amount of from 20% to 80% by weight;

b. Magnesium aluminometasilicate in an amount from 0.5% to 25% by weight;

c. One or more filler in an amount of from 5% to 70% by weight;

d. One or more binder in an amount of from 0.5% to 8% by weight;

e. One or more disintegrant in an amount of from 1% to 15% by weight;

f. One or more lubricant in an amount of from 0.5% to 10% by weight.

In a specific embodiment, bempedoic acid, a filler, a binder and a disintegrant are used intragranularly while magnesium aluminometasilicate, another filler, another disintegrant and a lubricant are used extragranularly.

In a second preferred embodiment, the pharmaceutical composition of the present invention contains the following ingredients, based on the total weight of the composition:

a. Bempedoic acid in an amount of from 20% to 80% by weight;

b. Magnesium aluminometasilicate in an amount from 0.5% to 25% by weight;

c. One or more filler in an amount of from 5% to 70% by weight;

d. One or more binder in an amount of from 0.5% to 8% by weight;

e. One or more disintegrant in an amount of from 1% to 15% by weight;

f. One or more lubricant in an amount of from 0.5% to 10% by weight;

g. Optionally, one or more glidant in an amount of from 0.2% to 10% by weight.

In a specific embodiment, Bempedoic acid, magnesium aluminometasilicate, a filler, a binder and a disintegrant are used intragranularly while another filler, another disintegrant, a lubricant and the optional glidant are used extragranularly.

The compositions of the present invention can be prepared by granulating the Bempedoic acid, magnesium aluminometasilicate and one or more pharmaceutically acceptable excipients, optionally followed by encapsulation or compression, using equipment and methods well-known to the skilled artisan.

In a preferred embodiment, the composition is prepared by granulation process and compressed into tablets. Granulation can be performed by a wet or dry process, wherein wet granulation, using water or organic solvents or mixtures thereof as granulation liquid, and dry granulation can be performed by processes known as slugging and/or roller compaction.

In a preferred embodiment, the composition is prepared by wet granulation process.

In one embodiment of the invention the composition is prepared by standard granulation process in which Bempedoic acid and one or more pharmaceutically acceptable excipients are granulated for a period from 7 to 20 minutes, preferably for around 15 minutes. The obtained granules are milled and magnesium aluminometasilicate and one or more pharmaceutically acceptable excipients are added extragranularly and blended. Finally, the mixture is compressed.

In another embodiment of the invention the composition is prepared by standard granulation process in which first Bempedoic acid is granulated with magnesium aluminometasilicate for a period from 15 to 30 minutes, preferably for around 15 minutes; one or more pharmaceutically acceptable excipients are further added and blended. The obtained granules are milled and one or more pharmaceutically acceptable excipients is added extragranularly and blended. Finally, the mixture is compressed.

The present invention also relates to a pharmaceutical composition in the form of tablet comprising granulates as described hereinabove

The pharmaceutical compositions described herein can be made using conventional methods and equipment well-known in the art.

The pharmaceutical compositions of the present invention show an in vitro dissolution profile wherein at least 75% of Bempedoic acid is released at fifteen minutes when the composition is subjected to a dissolution study in 900 ml HCl 0.1N (pH 6.6) using a USP apparatus II at 50 rpm at 37° C. Preferably, at least 80% of Bempedoic acid is released from the pharmaceutical composition at fifteen minutes. The pharmaceutical composition in accordance with the present invention is bioequivalent to the commercially available Bempedoic acid tablets (NILEMDO®/NEXLETOL®).

The present invention is illustrated by the following Examples.

### EXAMPLES

### Example 1

Formulation A

| INGREDIENTS | % | weight/tab (mg) |
|---|---|---|
| Intragranular composition | | |
| Bempedoic acid | 60.00% | 180.000 |
| L-Hydroxipropilcellulose | 4.00% | 12.000 |
| Lactose monohydrate | 9.33% | 28.000 |
| Sodium starch glycolate | 5.25% | 15.750 |
| Povidone | 2.00% | 6.000 |
| Extragranular composition | | |
| Magnesium aluminometasilicate | 1.17% | 3.500 |
| Microcrystalline cellulose | 15.00% | 45.000 |
| Sodium starch glycolate | 1.75% | 5.250 |
| Magnesium stearate | 1.50% | 4.500 |
| TABLET WEIGHT | 100.00% | 300.000 |
| OPADRY ® white 85F18422 | 3.00% | 9.000 |
| Total | 103.00% | 309.000 |

20 g of Povidone K30 was dissolved in water. 600 grams of bempedoic acid, 40 grams of hydroxipropilcellulose, 93.33 grams of lactose monohydrate and 52.5 grams of sodium starch glycolate were sieved through a 1.5 mm mesh size and blended in a bin blender for 5min at 20 rpm. Sieved powders are transferred to a high shear mixer and the solution containing the povidone K30 was added. A granulation was carried out. The wet granules were optionally sieved through 5 mm mesh and dried in a fluid bed at around 60° C. The same procedure was repeated twice, obtaining three sub-batches of granules. The three sub-batches were afterwards sieved through a 1.1 mm mesh size and transferred into an appropriate bin 10 blender. 35 g of magnesium aluminometasilicate were sieved through 0.8 mm mesh size and blended with the sieved granules for 30 min at 20 rpm. 450 g of microcrystalline cellulose and 52.5 g of sodium starch glycolate were de-agglomerated and added to the blender and the blend was mixed for 10 min at 20 rpm. 45 g of magnesium stearate was sieved and mixed with the blend in the bin blender for 3 min at 20 rpm.

The homogeneous blend was compressed under controlled humidity on a tablet press. The tablets were coated with Opadry® II until 3% of wt gain.

### Example 2

Formulation B

| Ingredients | % | Weight/tab (mg) |
|---|---|---|
| Intragranular composition | | |
| Bempedoic acid | 60.00 | 180.000 |
| Magnesium aluminometasilicate | 1.17 | 3.500 |
| L-Hydroxipropilcellulose | 4.00 | 12.000 |
| Lactose monohydrate | 9.33 | 28.000 |
| Sodium starch glycolate | 5.25 | 15.750 |
| Povidone | 2.00 | 6.000 |
| Extragranular composition | | |
| Microcrystalline cellulose | 15.00 | 45.000 |
| Sodium starch glycolate | 1.75 | 5.250 |
| Magnesium stearate | 1.50 | 4.500 |
| TABLET WEIGHT | 100.00 | 300.000 |
| OPADRY ® white 85F18422 | 3.00 | 9.000 |
| Total | 103.00 | 309.000 |

20 g of Povidone K30 was dissolved in water. 600 grams of Bempedoic acid and 11.67 g of magnesium aluminometasilicate were sieved through a 1.5 mm mesh size and blended in a bin blender for 30 min at 20 rpm. 40 grams of hydroxipropilcellulose, 93.33 grams of lactose monohydrate and 52.5 grams of sodium starch glycolate were sieved through a 1.5 mm mesh size and blended with the previous blend for 5 min at 20 rpm. Obtained blend was transferred to a high shear mixer and the solution containing the povidone K30 was added. A granulation was carried out. The wet granules were optionally sieved through 5 mm mesh and dried in a fluid bed at around 60°° C. The same procedure was repeated twice, obtaining three sub-batches of granules. The three sub-batches were afterwards sieved through a 1.1 mm mesh size and transferred into an appropriate bin blender. 450 g of microcrystalline cellulose and 52.5 g of sodium starch glycolate were de-agglomerated and added to the blender and the blend was mixed for 10 min at 20 rpm. 45 g of magnesium stearate was sieved and mixed with the blend in the bin blender for 3 min at 20 rpm.

The homogeneous blend was compressed under controlled humidity on a tablet press.

The tablets were coated with Opadry® II until 3% of wt gain.

### Example 3

Formulation C

| INGREDIENTS | % | weight/tab (mg) |
|---|---|---|
| Intragranular composition | | |
| Bempedoic acid | 60.00% | 180.000 |
| Magnesium aluminometasilicate | 1.17% | 3.500 |
| L-Hydroxipropilcellulose | 4.00% | 12.000 |
| Lactose monohydrate | 9.33% | 28.000 |
| Sodium starch glycolate | 5.25% | 15.750 |
| Extragranular composition | | |
| Microcrystalline cellulose | 16.50% | 49.500 |
| Colloidal silicon dioxide | 0.50% | 1.500 |
| Sodium starch glycolate | 1.75% | 5.250 |
| Magnesium stearate | 1.50% | 4.500 |
| TABLET WEIGHT | 100.00% | 300.000 |
| OPADRY ® white 85F18422 | 3.00% | 9.000 |
| Total | 103.00% | 309.000 |

600 grams of bempedoic acid and 11.67 g of magnesium aluminometasilicate were sieved through a 1.5 mm mesh size and blended in a bin blender for 30 min at 20 rpm. 40 grams of hydroxipropilcellulose, 93.33 grams of lactose monohydrate and 52.5 grams of sodium starch glycolate were sieved through a 1.5 mm mesh size and blended with the previous blend for 5 min at 20 rpm. Obtained blend was transferred to a high shear mixer and a water solution was added. A granulation was carried out. The wet granules were optionally sieved through 5 mm mesh and dried in a fluid bed at around 60° C. The same procedure was repeated twice, obtaining three sub-batches of granules. The three sub-batches were afterwards sieved through a 1.1 mm mesh size and transferred into an appropriate bin blender. 495 g of microcrystalline cellulose, 15 g of colloidal silicon dioxide and 52.5 g of sodium starch glycolate were de-agglomerated and added to the blender and the blend was mixed for 10 min at 20 rpm. 45 g of magnesium stearate was sieved and mixed with the blend in the bin blender for 3 min at 20 rpm.

The homogeneous blend was compressed under controlled humidity on a tablet press. The tablets were coated with Opadry® II until 3% of wt gain.

1. A pharmaceutical composition comprising bempedoic acid and magnesium aluminometasilicate.

2. The pharmaceutical composition according to claim 1, wherein the weight ratio of magnesium aluminometasilicate to bempedoic acid ranges from 1:2 to 1:120.

3. The pharmaceutical composition according to claim 1, wherein said bempedoic acid is present in an amount of from 20% to 80% by weight based on the total composition weight.

4. The pharmaceutical composition according to claim 1, wherein said magnesium aluminometasilicate is in an amount of from 0.5% to 25% by weight based on the total composition weight.

5. The pharmaceutical composition according to claim 1, wherein the composition comprises granules that contain said bempedoic acid.

6. The pharmaceutical composition according to claim 5, wherein said granules comprise the magnesium aluminometasilicate.

7. The pharmaceutical composition according to claim 1, comprising based on total weight of the composition:
a) bempedoiebempedoic acid in an amount of from 20% to 80% by weight;
b) magnesium aluminometasilicate in an amount from 0.5% to 25% by weight;
c) one or more filler in an amount of from 5% to 70% by weight;
d) one or more binder in an amount of from 0.5% to 8% by weight;
e) one or more disintegrant in an amount of from 1% to 15% by weight;
f) one or more lubricant in an amount of from 0.5% to 10% by weight.

8. The pharmaceutical composition according to claim 1 which further comprises based on total weight of the composition:
one or more glidant in an amount of from 0.2% to 10% by weight.

9. The pharmaceutical composition according to claim 7, wherein said filler is selected from the group comprising mannitol, sorbitol, microcrystalline cellulose, lactose, lactose monohydrate, phosphates, cellulose, hydroxypropyl cellulose, starch, pregelatinized starch, modified starch, sucrose, dextrose, dextrates, maltodextrin, xylitol, cyclodextrines, calcium phosphate, calcium sulfate and talc.

10. The pharmaceutical composition according to claim 7, wherein said binder is selected from the group comprising povidone, low substituted hydroxypropyl cellulose, hydroxypropyl methylcellulose, hydroxymethylpropylcellulose, sodium carboxyl methylcellulose, pregelatinized starch, starch, PEG and gelatin.

11. The pharmaceutical composition according to claim 7, wherein said disintegrant is selected from the group comprising crospovidone, sodium starch glycolate, croscarmellose sodium, natural starch, pregelatinized starch, sodium starch, methylcrystalline cellulose, methylcellulose, croscarmellose, crosslinked sodium carboxymethylcellulose, cross-linked croscarmellose, cross-linked polyvinilpyrrolidone, sodium alginate and gum.

12. The pharmaceutical composition according to claim 7, wherein said lubricant is selected from the group comprising magnesium stearate, calcium stearate, stearic acid, glyceryl behenate, hydrogenated vegetable oil, talc and sodium stearyl fumarate.

13. The pharmaceutical composition according to claim 5, wherein said composition is a tablet and said magnesium aluminometasilicate is extragranular.

14. The pharmaceutical composition according to claim 1, wherein the composition is a tablet and said bempedoic acid comprises 40% to 70% by weight and said magnesium aluminometasilicate comprises 0.5% to 10% by weight.

15. The pharmaceutical composition according to claim 14, wherein said tablet comprises granules and said bempedoic acid is present intragranularly.

17. The pharmaceutical composition according to claim 7, wherein said bempedoic acid is present intragranularly and said magnesium aluminometasilicate is present extragranularly.

18. The pharmaceutical composition according to claim 7, wherein said bempedoic acid and said magnesium aluminometasilicate are present intragranularly.

19. The pharmaceutical composition according to claim 8, wherein said bempedoic acid and said magnesium aluminometasilicate are present intragranularly and said glidant is present extragranularly.
